(19) United States
(12) Patent Application Publication
(10) Pub. No.: US 2014/0290663 A1
(43) Pub. Date: Oct. 2, 2014

(54) PATIENT INTERFACE DEVICE INCLUDING A MODULAR HUB INTERFACE

(71) Applicant: KONINKLIJKE PHILIPS N.V., Eindhoven (NL)

(72) Inventor: Justin Edward Rothermel, Monroeville, PA (US)

(21) Appl. No.: 14/351,305

(22) PCT Filed: Oct. 12, 2012
(86) PCT No.: PCT/IB2012/055558

§ 371 Date: Apr. 11, 2014

Related U.S. Application Data
(60) Provisional application No. 61/549,430, filed on Oct. 20, 2011.

(51) Int. Cl. A61M 16/06; A61M 16/08

(52) U.S. Cl. CPC A61M 16/0605; A61M 16/0816
USPC 128/206.24

(57) ABSTRACT

A patient interface device includes a cushion member having a first side, a second side, and a first outer perimeter. The first side is structured to form a seal with a patient's face for delivering a flow of breathing gas to the patient. A modular hub is attached to the second side of the cushion member. The modular hub includes a receiver and a second outer perimeter. The second outer perimeter of the modular hub is disposed inward of the first outer perimeter of the cushion member when the patient interface device is viewed from a front elevation perspective.

### CROSS-REFERENCE TO RELATED APPLICATIONS

This patent application claims the priority benefit under 35 U.S.C. §119(e) of U.S. Provisional Application No. 61/549,430 filed on Oct. 20, 2011, the contents of which are herein incorporated by reference.

### BACKGROUND OF THE INVENTION

1. Field of the Invention

The present invention relates to respiratory patient interface devices, and, in particular, to a respiratory patient interface device including a modular hub interface having a minimal profile.

2. Description of the Related Art

There are numerous situations where it is necessary or desirable to deliver a flow of breathing gas non-invasively to the airway of a patient, i.e., without intubating the patient or surgically inserting a tracheal tube in their esophagus. For example, it is known to ventilate a patient using a technique known as non-invasive ventilation. It is also known to deliver continuous positive airway pressure (CPAP) or variable airway pressure, which varies with the patient's respiratory cycle, to treat a medical disorder, such as sleep apnea syndrome, in particular, obstructive sleep apnea (OSA), or congestive heart failure.

Non-invasive ventilation and pressure support therapies involve the placement of a patient interface device including a mask component on the face of a patient. The mask component may be, without limitation, a nasal mask that covers the patient's nose, a nasal cushion that rests beneath the patient's nose (such as a “pillows” style nasal cushion having nasal prongs that are received within the patient's nares or a “cradle” style nasal cushion that rests beneath and covers the patient's nares), a nasal/oral mask that covers the nose and mouth, or a full face mask that covers the patient's face. The patient interface device interfaces the ventilator or pressure support device with the airway of the patient through tubing, so that a flow of breathing gas can be delivered from the pressure/flow generating device to the airway of the patient. It is known to maintain such devices on the face of a wearer by a headgear having one or more straps adapted to fit over/around the patient's head.

For such patient interface devices, a key engineering challenge is to balance patient comfort against mask stability. This is particularly true in the case of treatment of OSA, where such patient interface devices are typically worn for an extended period of time. Known mask assemblies, for example, typically include a cushion member designed to comfortably form a seal with the patient's face, and a hub which is joined to the cushion member and structured to receive the tubing or conduit. The hub is typically made of relatively hard material, whereas the cushion member is soft to promote patient comfort. However, known hubs tend to be excessively large, such that the hub extends outwardly from the patient's face farther than desired, making for an undesirably bulky assembly. The hub also extends laterally outwardly with respect to the cushion perimeter. If the hub extends too far, and/or if the hub-to-cushion member interface is not ideal, patient comfort is compromised. In addition, hub/cushion assemblies are often limited with respect to the type of elbow connections and/or frame members they can be employed with.

### SUMMARY OF THE INVENTION

Accordingly, it is an object of the present invention to provide a patient interface device that overcomes the shortcomings of conventional patient interface devices. This object is achieved according to one embodiment of the invention by providing a patient interface device including a modular hub having a minimal profile.

It is yet another object of the present invention to provide a method of delivering a flow of breathing gas that does not suffer from the disadvantages associated with conventional techniques. This object is achieved by providing a method that includes delivering a flow of breathing gas to a patient using a patient interface device having a cushion member and a modular hub with a minimal profile.

In one embodiment, a patient interface device is provided that includes a cushion member and a modular hub. The cushion member includes a first side, a second side, and a first outer perimeter. The first side is structured to form a seal with a patient's face for delivering a flow of breathing gas to the patient. The modular hub is attached to the second side of the cushion member, and includes a receiver and a second outer perimeter. The second outer perimeter of the modular hub is disposed inward of the first outer perimeter of the cushion member when the patient interface device is viewed from a front elevation perspective.

The patient interface may further include a frame member having a fluid coupling device. The receiver is structured to connect the modular hub to the fluid coupling device. The fluid coupling device may be an elbow, wherein the receiver of the modular hub is structured to provide a snap fit connection with the elbow, thereby removably securing the modular hub and the cushion member to the frame member.

In another embodiment, a method of delivering a flow of breathing gas to a patient is provided that includes generating the flow of breathing gas, delivering the flow of breathing gas to a patient interface device, and forming a seal between the patient's face and the first side of the cushion member. The patient interface device has a cushion member and a modular hub. The cushion member includes a first side, a second side, and a first outer perimeter. The modular hub is attached to the second side of the cushion member, and includes a receiver and a second outer perimeter. The second outer perimeter of the modular hub is disposed inward of the first outer perimeter of the cushion member when the patient interface device is viewed from a front elevation perspective.

These and other objects, features, and characteristics of the invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIG. 1 is a simplified view of a system adapted to provide a regimen of respiratory therapy to a patient according to one exemplary embodiment of the invention;

FIG. 2 is a front isometric view of a cushion member and modular hub assembly forming a part of the patient interface system of FIG. 1;

FIG. 3 is a side elevation view of the cushion member and modular hub assembly of FIG. 2;

FIG. 4 is a front elevation view of the cushion member and modular hub assembly of FIG. 2;

FIG. 5 is a bottom elevation view of the cushion member and modular hub assembly of FIG. 2;

FIG. 6 is a section view taken along line 6-6 of FIGS. 4; and

FIG. 7 is a section view taken along line 7-7 of FIG. 4.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “attached” and “directly coupled” mean that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

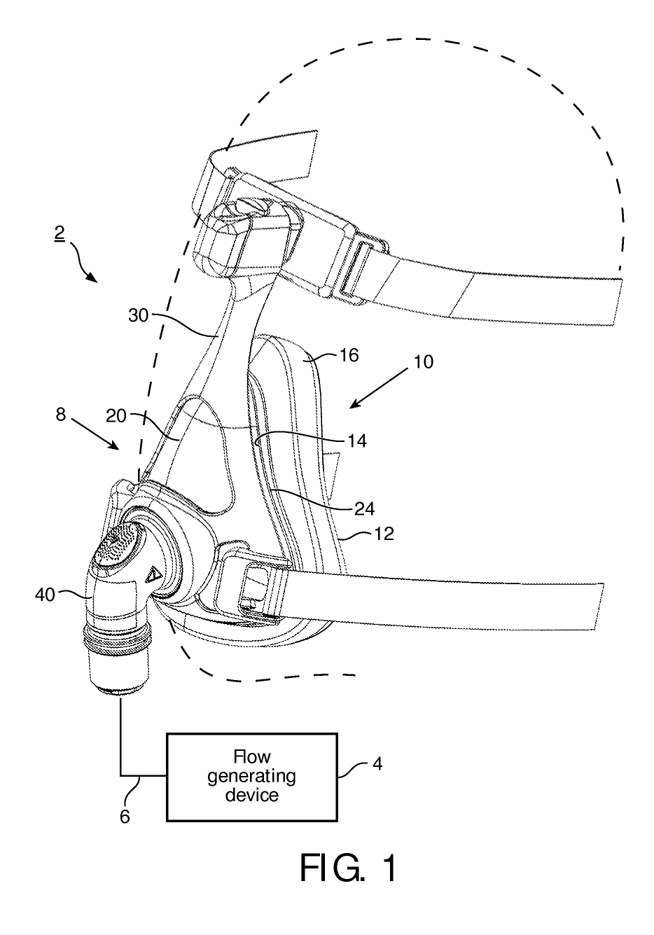

A system 2 adapted to provide a regimen of respiratory therapy to a patient according to one exemplary embodiment is generally shown in FIG. 1. System 2 includes a flow generating device 4, a delivery conduit 6, and a patient interface device 8. Flow generating device 4 is structured to generate a flow of breathing gas and may include, without limitation, ventilators, constant pressure support devices (such as a continuous positive airway pressure device, or CPAP device), variable pressure devices (e.g., BiPAP®, Bi-Flex®, C-Flex™, or A-Flex™ devices manufactured and distributed by Philips Respironics of Murrysville, Pa.), and auto-titration pressure support devices. Conduit 6 is structured to communicate the flow of breathing gas from flow generating device 4 to patient interface device 8 through conduit 6. Conduit 6 and patient interface device 8 are often collectively referred to as a patient circuit.

In FIG. 1, patient interface device 8 is a nasal/oral mask. However, other types of patient interface devices, such as, for example and without limitation, a full face mask, which facilitates the delivery of the flow of breathing gas to the airway of a patient, may be alternatively employed, without departing from the scope of the present invention.

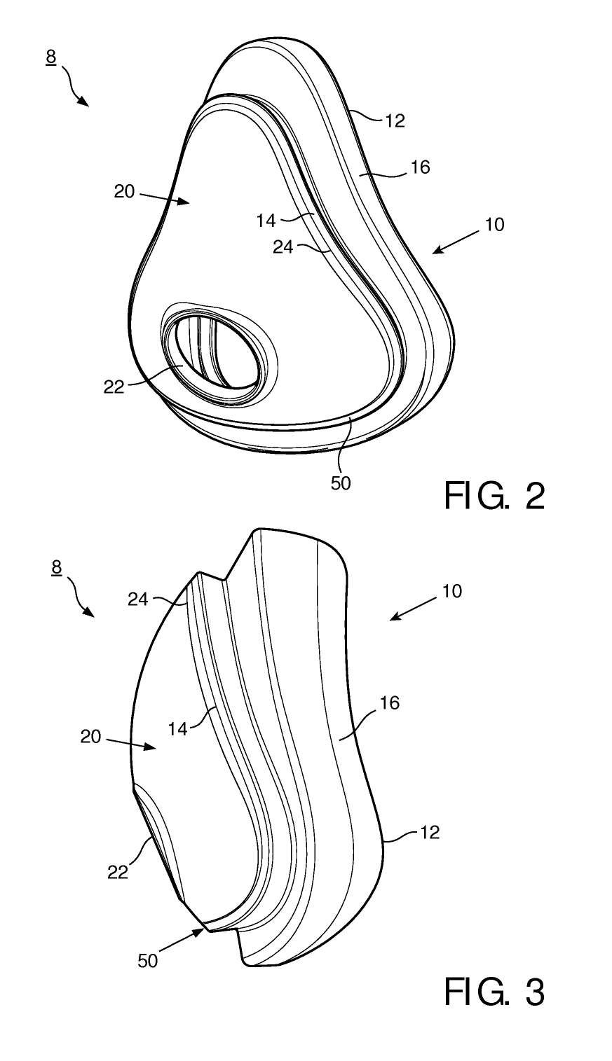

In accordance with one non-limiting example embodiment of the invention, shown and described with reference to FIGS. 1-7, patient interface device 8 includes a cushion member 10, having a first side 12, a second side 14, and a first perimeter 16. First side 12 is structured to form a seal with the patient's face (partially shown in simplified form in FIG. 1) for delivering a flow of breathing gas to the patient. A modular hub 20 is attached (e.g., without limitation, molded; glued or otherwise suitably bonded) to second side 14 of cushion member 10. As shown, for example, in FIGS. 2, 4 and 5, modular hub 20 includes a receiver 22 and a second outer perimeter 24.

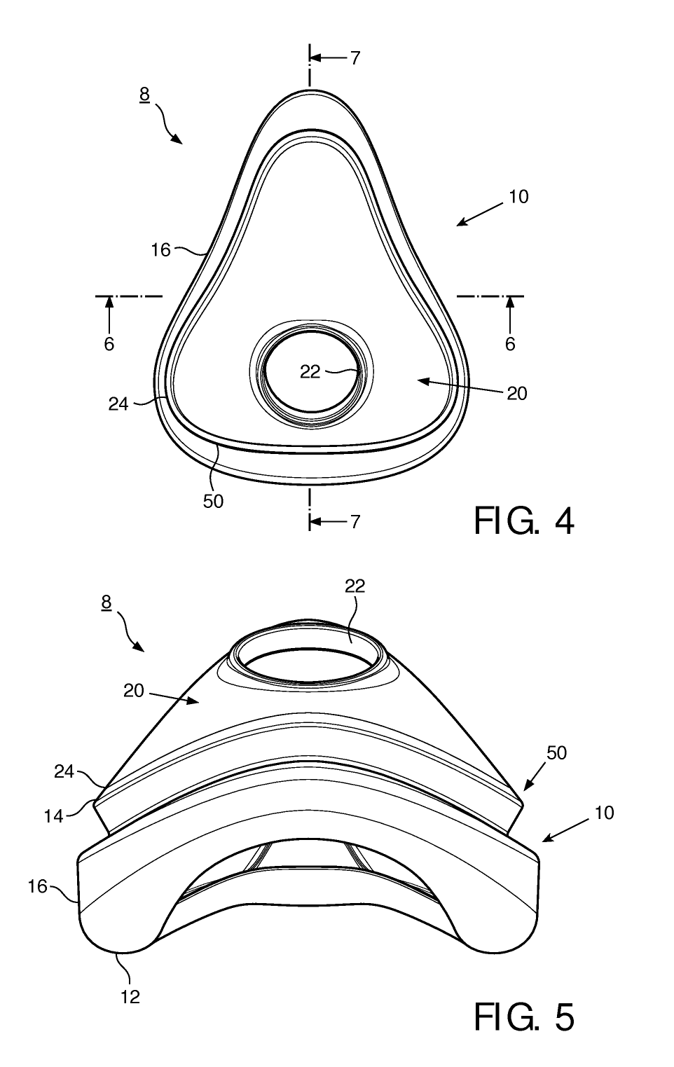

Referring to FIG. 4, it will be appreciated that second outer perimeter 24 of modular hub 20 is disposed inward of first outer perimeter 16 of cushion member 10, when patient interface device 8 is used from a front elevation respective, as shown. More specifically, modular hub 20 fits within outer perimeter 16 of cushion member 10, thereby reducing the lateral profile of the assembly. Among other benefits, this simplifies and improves installation and removal of patient interface device 8, for example, with respect to frame 30 and/or fluid coupling device 40, shown in the example of FIG. 1. The modular design of cushion member 10 and modular hub 20 enables patient interface device 8 to be readily employed with a wide variety of different frame members (e.g., without limitation, frame member 30 of FIG. 1) and/or fluid coupling devices (e.g., without limitation, elbow 40 of FIG. 1). In FIG. 1, fluid coupling device 40 is an elbow, wherein receiver 22 of modular hub 20 is structured to provide a snap fit connection with elbow 40, thereby removably securing modular hub 20 and cushion member 10 to frame member 30.

It will be appreciated, therefore, that the design of cushion member 10 and modular hub 20 of patient interface 8 is intended to have a minimum profile for providing maximum stability and comfort. More specifically, modular hub 20 does not extend laterally outwardly beyond outer perimeter 16 of cushion member 10 (best shown in the front elevation view of FIG. 4), and it also has a relatively low profile (e.g., height, when viewed from the bottom elevation view of FIG. 5). That is, modular hub 20 only extends or protrudes outwardly, with respect to second side 12 of cushion member 10, a relatively minimal distance. This allows for a patient interface design, wherein the patient interface 8 is closer to the patient's face than known patient interface designs (not shown).

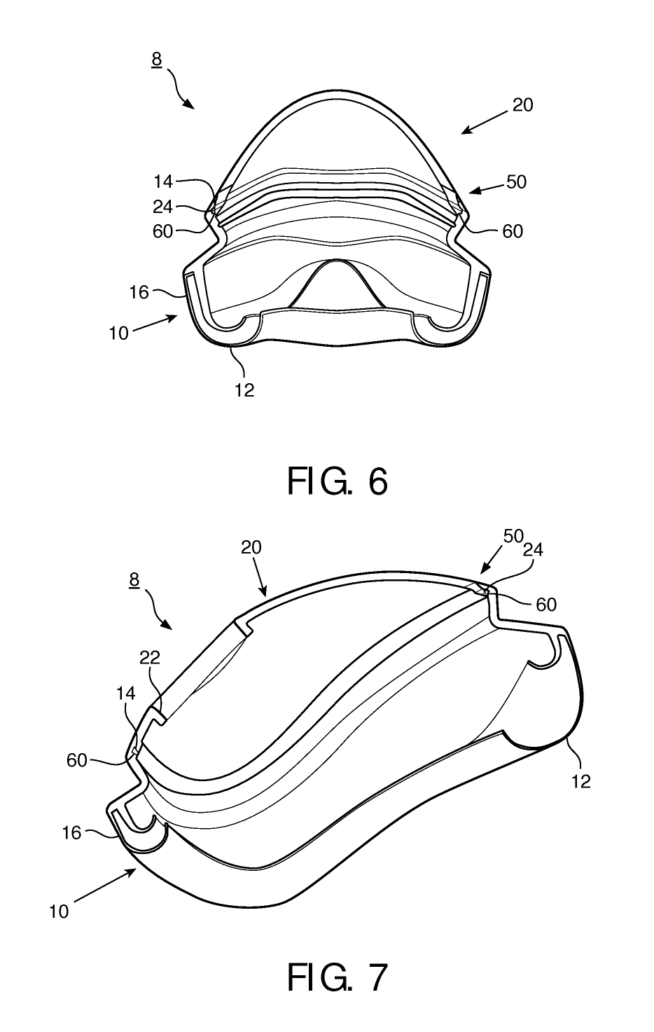

In addition to the aforementioned minimum profile attributes of patient interface 8, modular hub 20 is also provided with a relatively soft perimeter edge 60 (FIGS. 6 and 7), to further enhance comfort and safety while patient interface device 8 is in use. Specifically, modular hub 20 is made from a first material (e.g., without limitation, plastic) having a first hardness, and cushion member 10 is made from a second material (e.g., without limitation, rubber) having a second hardness, wherein the first hardness of modular hub 20 is greater or harder than the second hardness of cushion member 10. Accordingly, a portion of second side 14 of cushion member 10 preferably overlays edge 60 of second outer perimeter 24 of modular hub 20, as shown in FIGS. 6 and 7. In this manner, second outer perimeter 24 of modular hub 20 is wrapped or otherwise suitably covered with relatively soft material (e.g., without limitation, silicon). Thus, edge 60 is disposed at an inward location and is covered or otherwise suitably softened to enhance patient comfort.

Preferably, the interface 50 between modular hub 20 and cushion member 10 is generally seamless, as shown for example, in FIGS. 2-7. In other words, modular hub 20 and cushion 10 each have a substantially similar contour, or shape, at the location where modular hub 20 is attached to cushion member 10. Consequently, a very smooth and soft transition or interface 50 is provided between the two components. It will therefore be appreciated that this is what is meant by interface 50 being referred to as generally seamless. Such smooth or generally seamless nature of interface 50 will be further appreciated with reference to the section views of FIGS. 6 and 7.

Accordingly, patient interface device 8 provides a modular design, which can be readily employed with a wide variety of frame members (e.g., without limitation, frame member 30 of FIG. 1) and/or fluid coupling devices (e.g., without limitation, elbow 40 of FIG. 1). Patient interface device 8 also has a minimum profile such that it is disposed relatively close to patient's face, and has a soft outer perimeter 16 to enhance patient comfort and safety while in use.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. A patient interface device, comprising:
a cushion member including a first side, a second side, and a first outer perimeter, the first side being structured to form a seal with a patient's face for delivering a flow of breathing gas to the patient; and
a modular hub attached to the second side of the cushion member, the modular hub including a receiver and a second outer perimeter, wherein the second outer perimeter of the modular hub is disposed inward of the first outer perimeter of the cushion member when the patient interface device is viewed from a front elevation perspective.

2. The patient interface device of claim 1, further comprising a frame member having a fluid coupling device; and wherein the receiver is structured to connect the modular hub to the fluid coupling device.

3. The patient interface device of claim 2, wherein the fluid coupling device is an elbow; and wherein the receiver of the modular hub is structured to provide a snap fit connection with the elbow, thereby removably securing the modular hub and the cushion member to the frame member.

4. The patient interface device of claim 1, wherein the modular hub and the cushion each have a substantially similar contour at the location where the modular hub is attached to the cushion member.

5. The patient interface device of claim 1, wherein the interface between the modular hub and the cushion member is generally seamless.

6. The patient interface device of claim 1, wherein the modular hub is made from a first material having a first hardness; wherein the cushion member is made from a second material having a second hardness; and wherein the first hardness is greater than the second hardness.

7. The patient interface device of claim 6, wherein the second outer perimeter of the modular hub is wrapped in silicon.

8. The patient interface device of claim 1, wherein the second outer perimeter of modular hub defines an edge; and wherein a portion of the second side of the cushion member overlays the edge.

9. A method of delivering a flow of breathing gas to a patient, comprising:
generating the flow of breathing gas;
delivering the flow of breathing gas to a patient interface device, the patient interface device having a cushion member and a modular hub, the cushion member including a first side, a second side, and a first outer perimeter, the modular hub being attached to the second side of the cushion member, the modular hub including a receiver and a second outer perimeter, wherein the entire second outer perimeter of the modular hub is disposed inward of the first outer perimeter of the cushion member when the patient interface device is viewed from a front elevation perspective; and
forming a seal between the patient's face and the first side of the cushion member.

10. The method according to claim 9, wherein the patient interface device further includes a frame member having a fluid coupling device; and wherein the receiver is structured to connect the modular hub to the fluid coupling device.

11. The method according to claim 10, wherein the fluid coupling device is an elbow; and wherein the receiver of the modular hub is structured to provide a snap fit connection with the elbow, thereby removably securing the modular hub and the cushion member to the frame member.

12. The method according to claim 9, wherein the modular hub and the cushion each have a substantially similar contour at the location where the modular hub is attached to the cushion member.

13. The method according to claim 9, wherein the interface between the modular hub and the cushion member is generally seamless.

14. The method according to claim 9, wherein the modular hub is made from a first material having a first hardness; wherein the cushion member is made from a second material having a second hardness; and wherein the first hardness is greater than the second hardness.

15. The method according to claim 14, wherein the second outer perimeter of the modular hub is wrapped in silicon.

16. The method of claim 9, wherein the second outer perimeter of modular hub defines an edge; and wherein a portion of the second side of the cushion member overlays the edge.
